(19) United States
(12) Patent Application Publication
(10) Pub. No.: US 2026/0159852 A1
(43) Pub. Date: Jun. 11, 2026

(54) ACTIVATED FROZEN EUKARYOTIC CELL TRANSFECTION SYSTEMS AND RELATED METHODS

(71) Applicants: Florian M. Wurm, 1820 Montreux (CH); 1820 Montreux J. Wurm, 1820 Montreux (CH)

(72) Inventors: Florian M. Wurm, 1820 Montreux (CH); 1820 Montreux J. Wurm, 1820 Montreux (CH)

(21) Appl. No.: 19/412,680

(22) Filed: Dec. 8, 2025

Related U.S. Application Data
(60) Provisional application No. 63/729,182, filed on Dec. 6, 2024.

(51) Int. Cl. C12N 15/85; C12N 5/00; C12N 5/071; C12N 5/10

(52) U.S. Cl. CPC C12N 15/85; C12N 5/0682; C12N 5/10; C12N 5/525; C12N 2510/04

(57) ABSTRACT

The present invention provides streamlined eukaryotic cell transfection systems and media, as well as related methods and kits. The activated frozen cell transfection systems, media, and methods of the present invention eliminate the need for specialized equipment and avoid the time commitment required to pre-culture the cells and to prepare cells with a special medium and unique handling procedures for transfection. In particular, the systems and related methods afford the ability for streamlining transfection while retaining robust transfection efficiencies. Furthermore, the activated frozen cell transfection systems, media, and methods of the present invention for transfecting eukaryotic cells makes transfection available for both high throughput approaches in small scale and/or for large scale transfection without requiring specialized equipment or requiring a pre-culture step subsequent to thawing.

### RELATED APPLICATIONS

This application claims priority to U.S. Provisional Patent Application No. 63/729,182, filed on Dec. 6, 2024; the entirety of which is incorporated herein by reference.

### BACKGROUND OF THE INVENTION

Many of today's biologic drug therapies (biologicals) are produced using genetically transformed (transgenic or recombinant) eukaryotic cells, for example, using recombinant mammalian cells. In fact, in terms of specialized reagents and equipment, the market for the generation of transgenic or recombinant eukaryotic cells has exceed one billion dollars in sales. Biologicals of interest in this context are recombinant proteins, such as antibodies, enzymes, fusion molecules and the like, as well as more complex products such as viral surface proteins, virus-like particles, retrovirus like vectors, and any other protein, DNA or RNA.

The putative process of generating transformed cells to express a gene of interest. e.g., recombinant eukaryotic cells, is a multi-step, multi-day series of processes, beginning with proper design of the nucleic acid construct to be used and choice of an appropriate cell line. Subsequent steps involve the growth of the selected cells in culture for numerous days followed by the introduction of the nucleic acid through the cellular membrane into the cells. The nucleic acid introduction process is typically called transfection. Following successful transfection, the cells are grown under conditions to express the gene or genes encoded by the nucleic acid, and the gene products, e.g., protein products, are then harvested for use.

Despite the passage of over fifty years since the first successful mammalian cell transfection, the introduction of nucleic acid into eukaryotic cells, such as mammalian cells, has remained a complex, time consuming, and highly error-prone procedure that rarely provides predictable results. Numerous chemical, physical, and viral methods requiring specific reagents and equipment have been employed. The disadvantages of these methods are numerous and well known to the skilled artisan, including being labor and time intensive, as well as requiring specialized equipment and techniques, and cumbersome preparation of cells, especially the long and difficult-to-control pre-culture of cells.

Thus, there is a need in the art for a streamlined approach to transfecting eukaryotic cells (such as mammalian, insect, or fish cells) with a desired nucleic acid that avoids the need for specialized equipment and the time commitment to pre-culture the cells.

### SUMMARY OF THE INVENTION

Accordingly, the present invention provides streamlined eukaryotic cell transfection systems and media, as well as related methods and kits. The activated frozen cell transfection systems, media, and methods of the present invention eliminate the need for specialized equipment and avoid the time commitment required to pre-culture the cells. In particular, the systems and related methods afford the ability for streamlining transfection while retaining robust transfection efficiencies. Furthermore, the activated frozen cell transfection systems, media, and methods of the present invention for transfecting eukaryotic cells makes transfection available for both high throughput approaches in small scale and/or for large scale transfection without requiring specialized equipment or requiring a pre-culture step subsequent to thawing.

As such, one aspect of the invention provides an activated frozen cell transfection system (AFCT-system) that is configured for transfection of a nucleic acid into a eukaryotic cell immediately upon thaw and without a pre-culture step comprising: a cell transfection medium with a cryopreservant overload ratio; and a eukaryotic cell for combination with said cell transfection medium to produce cell-supplemented cell transfection medium, wherein the cell transfection medium combined with the eukaryotic cell are frozen.

Another aspect of the present invention provides an activation medium for preparing an activated frozen cell transfection system (AFCT-system) that is configured for transfection of a nucleic acid into a eukaryotic cell immediately upon thaw and without a pre-culture step comprising: a cell transfection medium with a cryopreservant overload ratio; and a eukaryotic cell for combination with said cell transfection medium to produce cell-supplemented cell transfection medium, wherein the cell transfection medium combined with the eukaryotic cell are ready for freezing.

Another aspect of the present invention provides a method of transfecting eukaryotic cells. The method comprises the step of obtaining an activated frozen cell transfection system (AFCT-system) that is configured for transfection of a nucleic acid into a eukaryotic cell immediately upon thaw and without a pre-culture step comprising: a cell transfection medium with a cryopreservant overload ratio; and a eukaryotic cell for combination with said cell transfection medium to produce cell-supplemented cell transfection medium, wherein the cell transfection medium combined with the eukaryotic cell are frozen. The method further comprises the steps of thawing the activated frozen cell transfection system; and combining the thawed activated frozen cell transfection system with exogenous nucleic acid without a pre-culture step, such that said exogenous nucleic acid is transfected into said eukaryotic cells immediately upon thaw and without a pre-culture step.

In another aspect, the present invention provides a method of preparing an activated frozen cell transfection system (AFCT-system). The method comprises the step of obtaining an activation medium that is configured for transfection of a nucleic acid into a eukaryotic cell immediately upon thaw and without a pre-culture step comprising: a cell transfection medium with a cryopreservant overload ratio; and a eukaryotic cell for combination with said cell transfection medium to produce cell-supplemented cell transfection medium. The method further comprises the step of freezing the cell transfection medium combined with the eukaryotic cell;

Another aspect of the present invention provides a kit for transfection of eukaryotic cells comprising: an activated frozen cell transfection system (AFCT-system) of the present invention; and a nucleic acid that is a DNA vector compatible with said eukaryotic cells.

### BRIEF DESCRIPTION OF THE FIGURES

Advantages of the present invention will be apparent from the following detailed description, which description should be considered in combination with the accompanying figures, which are not intended to limit the scope of the invention in any way.

FIG. 1 shows a simplified representation of the invention in comparison to the standard procedure of transient transfection and production, depicting (A) diagram for transfection of the present invention, and (B) establishment of a seed-train culture over several days and weeks until availability of a sufficient and healthy cell biomass is provided at the right time for engaging into transfections, where the steps which are avoided upon using the invention are indicated by crossed-out activities.

FIG. 2 depicts the results in terms of production yields (μg/mL) from FT-CHO4Tx™ cells, frozen in FTM with different cell densities and with the corresponding adjustments of the final transfection/production volume.

FIG. 3 depicts results in terms of product yields from transfections under reduction of DNA by 75% while also modifying cell number in frozen vials (black) and adjusting the transfected volume for production. Control transfections with non-frozen cells (grey).

FIG. 4 depicts results in product yield upon scale-up of the method to the 300 mL scale in TubeSpin® bioreactor 600 (TPP Birsfelden, Switzerland). A control transfection is from non-frozen cells at 10 mL scale in 50 mL TubeSpin bioreactor. One 30 mL Cyrovault provides cells for one transfection at a volume of 300 mL.

FIG. 5 depicts the storage stabilities of frozen cells at −80° C. for transfection after thaw. All transfections at 10 mL scale. A) 30 days: Transfections were executed with a reduction of DNA by 75% except in one control transfection. Productivity after 14 days in protein concentration in the supernatant. B) 180 days: Transfection efficiency as measured by GFP expression, day 3. Transfections were done as triplicates.

### DETAILED DESCRIPTION OF THE INVENTION

Despite existing efforts in the advancement of protein production, the known/existing efforts have yielded few significant improvements to transfection efficiencies and transfection reproducibility, especially with respect to reduction of the complexities of existing processes for transfection and the specialized equipment required for transfection. Existing standard freezing media usually have medium from a prior executed cell culture (up to 50%), which are derived from the supernatants of cultures after several days of exposure to cells for about 3-4 days. Such standard freezing media is not capable of transfection upon thaw, and requires extraction and replacement, as well as requisite well-defined carefully executed pre-culture steps before transfection is possible. In contrast, the activated frozen cell transfection systems, media, and methods of the present invention provide robust, streamlined, reproducible, predictable, user-friendly systems, media, and methods without requiring media exchange, specialized equipment or requiring a pre-culture step subsequent to thawing. In essence, the activated frozen cell transfection systems, media, and methods of the present invention offer instant transfection upon thawing from frozen cells.

The activated frozen cell transfection systems, media, and methods of the present invention eliminate the need for specialized equipment and avoid the time commitment required to pre-culture the cells and to prepare cells with a special medium and unique handling procedures for transfection. In particular, the systems, media, and related methods afford the ability for streamlining transfection while retaining robust transfection efficiencies. Furthermore, the activated frozen cell transfection systems, media, and methods of the present invention for transfecting eukaryotic cells makes transfection available for both high throughput approaches and/or for large scale transfection without requiring specialized equipment or requiring a pre-culture step subsequent to thawing.

The present invention, including systems, media, and methods will be described with reference to the following definitions that, for convenience, are set forth below. Unless otherwise specified, the below terms used herein are defined as follows:

### I. Definitions

As used herein, the terms “a,” “an,” “the” and similar terms used in the context of the present invention (especially in the context of the claims) are to be construed to cover both the singular and plural unless otherwise indicated herein or clearly contradicted by the context.

The term “about” is used herein in reference to the degree or extent of the term which it modifies, and that such extent may be 100% or near to but not exactly 100% of the modified term; industry accepted standards will assist in defining the quantitative aspects of how “near” 100% is defined. In particular embodiments, the term “about” indicates ±2%, ±1% or ±0.5%. In a particular embodiment, for example, the language “about 19%” would be exactly 19%; and in an alternative particular embodiment, for example, the language “about 19%” would mean 19%±2%, 19%±1%, or 19%±0.5%.

The term “activated” is used herein to describe the characterization of a frozen medium that is configured for transfection of a nucleic acid into a eukaryotic cell immediately upon thaw and without a pre-culture step. Such frozen medium is activated using a cryopreservant overload ratio, e.g., DMSO overload ratio. In certain embodiments, this activated medium does not need medium exchange prior to transfection (subsequent to thawing).

The term “activation” is used herein to describe the characterization of a medium that is capable of being frozen and then mediating transfection of a nucleic acid into a eukaryotic cell immediately upon thaw and without a pre-culture step. Such activation medium, once frozen may be used to create activated frozen medium, by using a cryopreservant overload ratio, e.g., DMSO overload ratio.

The language “cryopreservant” is art recognized, and is used herein to describe cryoprotectants or chemicals added to transfection media to prevent damaging ice crystals from forming inside cells. In certain embodiments, the cryopreservant is selected from one or more of acetamide, albumin, ammonium acetate, choline magnesium chloride sodium bromide, diethyl glycol, dimethylacetamide, dimethylsulfoxide, ethanol, erythritol EG, glycerol, glucose, formamide, glycerophosphate, proline, pyridine-N-oxide, propylene glycol, ribose, serine, sodium chloride, sodium bromide, sodium iodide, sodium sulfate, sorbitol, sucrose, triethylene glycol, trimethylamine, acetate, xylose, and valine. In particular embodiments, the cryopreservant is selected from DMSO, glycerol, sucrose, or any combination thereof. In specific embodiments, the cryopreservant is DMSO, e.g., at least 10% DMSO. In specific embodiments, the cryopreservant is glycerol, e.g., at least 20% glycerol. In specific embodiments, the cryopreservant is sucrose, e.g., at least 30% sucrose.

The language “DMSO overload ratio” is used herein to describe media used herein that comprises 10% or greater DMSO, i.e., at least 10% DMSO. Such overload ratio offers DMSO at the proper concentrations that ensure that the media and frozen cell transfection systems of the present invention remain activated upon thawing, and therefore do not require pre-culturing prior to use in transfection.

The term “eukaryotic” is well understood in the art and used herein to describe cells that contain a nucleus, e.g., eukaryotic cells. Eukaryotic cells include, but are not limited to, mammalian cells, e.g., human cells or animal cells, insect cells, fish cells, plant cells, fungal cells, and avian cells.

The term “exogenous nucleic acid” is used herein to describe that nucleic acid (e.g., DNA or RNA) which is introduced into a cell (e.g., a eukaryotic cell or a prokaryotic cell). The exogenous nucleic acid may have a nucleotide sequence that is 0% to 100% identical to, or complementary to, a DNA or RNA sequence already existing in the recipient cell.

The term “frozen” is art-recognized, and used herein to describe a medium that has been subjected to temperatures sufficient to freeze the entire medium, e.g., −80° C. In certain embodiments, the frozen medium is capable of being stored long-term (e.g., 30 days or more, e.g., 60 days or more, e.g., 90 days or more, e.g., 180 days or more, or e.g., 350 days or more) while retaining the capacity to mediate the transfection of nucleic acid into a recipient eukaryotic cell, e.g., a mammalian cell, e.g., a human cell or an animal cell.

The term “highly-efficient” is used herein to describe the advantages of certain eukaryotic cell transfection system and methods of the present invention, e.g., that are further pre-conditioned with a positively charged polymer that is not associated with nucleic acid, and which include one or more of the following advantages: (1) an increase in the percentage of cells successfully transfected as compared with existing methodologies/systems, e.g., 30% or greater of cells transfected, e.g., 50% or greater of cells transfected, e.g., 75% or greater of cells transfected, or e.g., 99% or greater of cells transfected; (2) an increase in the amount of product recovered from successfully transfected cells as compared with existing methodologies/systems, e.g., at least a 2-fold increase, e.g., at least a 5-fold increase, e.g., at least a 10-fold increase, e.g., at least a 20-fold increase, e.g., at least a 50-fold increase in product recovery, e.g., at least a 100-fold increase, or e.g., at least a 500-fold increase; (3) increased cell viability post-transfection as compared with existing methodologies/systems, e.g., 50% or greater cell viability, e.g., 60% or greater cell viability, e.g., 70% or greater cell viability, e.g., 80% or greater cell viability, e.g., 90% or greater cell viability, or e.g., 99% or greater cell viability; (4) an increase in cell number (in cells/ml) after transfection during the production phase as compared with existing methodologies/systems, e.g., a 10% or greater number of cells, e.g., a 20% or greater number of cells, e.g., a 30% or greater number of cells, e.g., a 50% or greater number of cells, e.g., a 75% or greater number of cells, e.g., a 100% or greater number of cells, e.g., a 150% or greater number of cells, e.g., a 300% or greater number of cells, e.g., a 500% or greater number of cells; (5) a higher reproducibility of transfections within an experiment as compared with existing methodologies/systems, for example, intra-experimental reproducibility (i.e., when multiple transfection are executed with the same seed cell culture), or for example, inter-experimental reproducibility (i.e., when done with seed cell cultures from different sources or when done on different days); and (6) elimination of laborious nucleic acid protection procedures as compared with existing methodologies/systems including, but not limited to, encapsulation or association of nucleic acid with or in liposomes, dendrimers, cyclodextrins, or cationic polymers, e.g., DEAE-dextran, e.g., polyethylenimine, e.g., polylysine and the like, nano-particles, metals, formation of calcium phosphate-DNA complexes, use of viral carrier vectors, and the like.

The term “mammalian cell” is also well understood in the art and is used herein to describe any primary cell, primary cell culture, or cultured cell line derived from a mammal. The term “animal cell” is also well understood in the art and is used herein to describe any primary cell or cultivated cell derived from a mammalian or non-mammalian species, such as fish, reptile or bird species, including cells from non-vertebrate species such as metazoans e.g., eukaryotic species with more than one cell).

The term “nucleic acid” is known in the art and used herein to described natural or synthetic polymers of deoxyribonucleic acid (DNA) (e.g., single-stranded or double-stranded), or ribonucleic acid (RNA), or polymers comprising a mix of both deoxyribonucleotides and ribonucleotides (DNA-RNA hybrids), peptide nucleic acid (PNA), or locked nucleic acid (LNA), and the like. As used herein, the term encompasses all forms and types of DNA, RNA, DNA-RNA hybrids, PNA or LNA contemplated for transfection into eukaryotic cells. In certain embodiments, the term “nucleic acid” is used synonymously with “exogenous nucleic acid.”

The language “overload ratio” is used herein to describe media used herein that comprises an amount/ratio that is significantly greater, i.e., overloaded, as compared with known uses of cryopreservant in transfections, e.g., at least 10% DMSO in certain examples of the present invention as compared to the maximum of known transfections (e.g., the present invention utilizes greater than 3× the maximum considered ratio where cell transfection is dramatically reduced in prior systems). Such overload ratio offers the cryopreservant at the identified, but unexpected, concentrations that ensure that the media and frozen cell transfection systems of the present invention remain activated upon thawing, and therefore do not require pre-culturing prior to use in transfection.

The term “polyethylenimine” (PEI) is known in the art and used herein to describe a polymer of repeating units composed of an amine group and two carbon aliphatic spacers, e.g., (C₂H₅N)ₙ. Due to the polymeric nature of PEI, polymers may range in molecular weight from about 800 Da to about 25 KDa to 1000 KDa or more. PEI polymers may be acylated or deacylated. Polymers of PEI may be linear, for example,

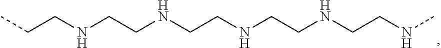

or branched, for example,

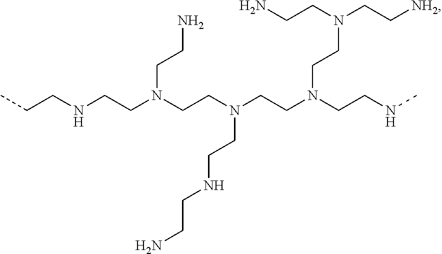

where the dashed lines represent the locations where the monomeric unit connects to another monomeric unit in the polymeric chain. These polymers can also be hyperbranched and/or dendritic. In certain embodiments, a given amount of a PEI polymer, e.g., a PEI polymer of 25 KDa, is polydisperse, i.e., comprises a range of molecules of different molecular weights having an average molecular weight of 25 KDa. In certain embodiments, identification of a molecular weight of a PEI polymer in this disclosure of the invention is to be understood to be of polydispersed character where the synthesis of PEI polymer is not uniform and may have produced a distribution of polymers with an “averaged” molecular weight.

The term “pre-conditioned” is used herein to describe the characteristic of a combination of two or more components which have been treated, conditioned or exposed to another agent or component (e.g., a pre-conditioning agent), prior to the introduction of one or more additional items (e.g., a nucleic acid, a compound, a solution, or a cell). For example, in certain embodiments, the cell transfection media of the invention are “pre-conditioned”, that is pre-mixed with or pre-exposed to polyethylenimine prior to exposure of the media to another compound or solution, e.g., to eukaryotic cells targeted for transfection, or e.g., to purified, unencumbered exogenous nucleic acid.

The language “purified, unencumbered” is used herein to describe the form and condition of exogenous nucleic acid having the characteristic of being free from compounds, molecules, chemicals, and treatments usually employed in eukaryotic cell transfections, and which may be required to facilitate transfection in traditional methodology. For example, the purified, unencumbered nucleic acid utilized in the systems, media, and methods of the present invention are free of (e.g., essentially free of), liposomes, dendrimers, cyclodextrins, or cationic polymers, e.g., DEAE-dextran, polylysine, polyethylenimine and the like, nano-particles, metals, formation of calcium phosphate-DNA complexes, use of viral carrier vectors, viral protein capsules, and the like. Purified, unencumbered exogenous nucleic acids may exist in an essentially solvent-free form, e.g., as a pure, precipitated pellet of material, or dissolved in an aqueous solution such as purified water, or TE buffer, e.g., 10 mM Tris 1 mM EDTA buffer, and the like.

The term “recipient cell” is used herein to describe a cell, e.g., a mammalian cell (e.g., a CHO cell), that is subjected to a transfection process, e.g., the highly-efficient method of nucleic acid introduction into eukaryotic cells of the invention, and is the first and direct recipient of the exogenous nucleic acid, e.g., exogenous DNA.

The term “species” is used herein to describe related variants of a particular cell type. For example, the first Chinese hamster ovary cells were isolated and immortalized in the late 1950's; since that time numerous variations or species of the original CHO cells, CHO-ori, have arisen, such as CHO-K1, CHO-S, CHO-DXB11, CHO-DG 44 and others. As used herein, CHO encompasses all species of CHO cells referred to in the art as a type of CHO cell, e.g., CHO-K1, CHO-S, CHO-DG 44, CHO-DXB11, CHOExpress®, and CHO4Tx® are all examples of species of CHO cells.

The term “transfection” is known in the art and used herein to describe the deliberate introduction of nucleic acids, e.g., exogenous nucleic acids, e.g., exogenous DNA, into eukaryotic cells. Eukaryotic cells may be transiently transfected or stably transfected. Transiently transfected eukaryotic cells take up and express exogenous nucleic acids, e.g., exogenous DNA, where the nucleic acid e.g., exogenous DNA, is generally not incorporated into the genome of the transfected cell. In contrast, for stable transfections, the exogenous nucleic acid, e.g., exogenous DNA, is incorporated into, e.g., integrated into, the genome of a stably transfected recipient cell; the exogenous nucleic acid, e.g., exogenous DNA, is replicated each time the genome is replicated prior to nuclear and/or cell division, and is passed through to multiple generation of cells, e.g., daughter cells. Stably transfected, eukaryotic cells may also be referred to as recombinant cells.

### II. Activated Frozen Cell Transfection Systems and Media of the Invention

The present invention offers activated frozen cell transfection systems, for example, created from activation medium afford transfection ready cells, e.g., CHO cells, that do not require pre-culturing before transfection. In certain embodiments these transfection ready cells are under cGMP (e.g., fully tested and released), and can go directly into a cGMP facility for generation of stable recombinant protein expressing cell populations (e.g., after selection with a resistance component that conveys survival under selection conditions) and for the transient transfection for direct production of proteins.

As such, one embodiment of the present invention provides an activation medium for preparing an activated frozen cell transfection system (AFCT-system) that is configured for transfection of a nucleic acid into a eukaryotic cell immediately upon thaw and without a pre-culture step comprising
a cell transfection medium with a cryopreservant overload ratio (e.g., at least 10% DMSO); and
a eukaryotic cell for combination with said cell transfection medium to produce cell-supplemented cell transfection medium,
   wherein the cell transfection medium combined with the eukaryotic cell are ready for freezing. In certain embodiments, the cryopreservant is selected from one or more of acetamide, albumin, ammonium acetate, choline magnesium chloride sodium bromide, diethyl glycol, dimethylacetamide, dimethylsulfoxide, ethanol, erythritol EG, glycerol, glucose, formamide, glycerophosphate, proline, pyridine-N-oxide, propylene glycol, ribose, serine, sodium chloride, sodium bromide, sodium iodide, sodium sulfate, sorbitol, sucrose, triethylene glycol, trimethylamine, acetate, xylose, and valine. In particular embodiments, the cryopreservant is DMSO.

As such, one embodiment of the present invention provides an activation medium for preparing an activated frozen cell transfection system (AFCT-system) that is configured for transfection of a nucleic acid into a eukaryotic cell immediately upon thaw and without a pre-culture step comprising
a cell transfection medium with a cryopreservant overload ratio, e.g., DMSO overload ratio (e.g., at least 10% DMSO); and
a eukaryotic cell for combination with said cell transfection medium to produce cell-supplemented cell transfection medium,
   wherein the cell transfection medium combined with the eukaryotic cell are ready for freezing.

Another embodiment of the present invention provides an activated frozen cell transfection system (AFCT-system) that is configured for transfection of a nucleic acid into a eukaryotic cell immediately upon thaw and without a pre-culture step comprising
a cell transfection medium with a cryopreservant overload ratio, e.g., DMSO overload ratio (e.g., at least 10% DMSO); and
a eukaryotic cell for combination with said cell transfection medium to produce cell-supplemented cell transfection medium,
   wherein the cell transfection medium combined with the eukaryotic cell are frozen. The systems of the present invention have the capability to mediate transfection of nucleic acid into a eukaryotic cell; however, this does not require actual mediation to have such potential or ability to mediate.

Another embodiment of the present invention provides an activated frozen cell transfection system (AFCT-system) that is configured for transfection of a nucleic acid into a eukaryotic cell immediately upon thaw and without a pre-culture step comprising
a cell transfection medium with a cryopreservant overload ratio (e.g., at least 10% DMSO); and
a eukaryotic cell for combination with said cell transfection medium to produce cell-supplemented cell transfection medium,
   wherein the cell transfection medium combined with the eukaryotic cell are frozen. The systems of the present invention have the capability to mediate transfection of nucleic acid into a eukaryotic cell; however, this does not require actual mediation to have such potential or ability to mediate. In certain embodiments, the cryopreservant is selected from one or more of acetamide, albumin, ammonium acetate, choline magnesium chloride sodium bromide, diethyl glycol, dimethylacetamide, dimethylsulfoxide, ethanol, erythritol EG, glycerol, glucose, formamide, glycerophosphate, proline, pyridine-N-oxide, propylene glycol, ribose, serine, sodium chloride, sodium bromide, sodium iodide, sodium sulfate, sorbitol, sucrose, triethylene glycol, trimethylamine, acetate, xylose, and valine. In particular embodiments, the cryopreservant is DMSO.

In certain embodiments of the present invention, the nucleic acid is a purified, unencumbered exogenous nucleic acid. In certain embodiments the purified, unencumbered exogenous nucleic acid for combination with the cell-supplemented pre-conditioned cell transfection medium is free from the components selected from the group consisting of proteins, cationic lipids, cationic polymers, liposome forming components, calcium phosphates, calcium chloride, nano-particles, metals, polymeric gene carriers, dendrimers, and cyclodextrins.

In certain embodiments of the present invention, the nucleic acid is purified, unencumbered exogenous nucleic acid, and the system is pre-conditioned with a positively charged polymer that is not associated with nucleic acid and is capable of mediating transfection of the purified, unencumbered exogenous nucleic acid into the eukaryotic cell.

In certain embodiments of the present invention, the system or medium may be pre-conditioned with a positively charged polymer that is not associated with nucleic acid and is capable of mediating transfection of purified, unencumbered exogenous nucleic acid into the eukaryotic cell. In particular embodiments, the positively charged polymer may be incorporated into the activation medium or into the activated frozen cell transfection system. In particular embodiments, the positively charged polymer may be added to the activated frozen cell transfection system after thawing.

In certain embodiments of the present invention, cell transfection medium comprises:
5-200 mg/L CaCl₂) (anhydrous);
15-70 mg/L MgCl₂ (anhydrous);
0-0.08 mg/L Fe(NO₃)₃ 9H₂O;
20-110 mg/L MgSO₄ (anhydrous);
30-100 mg/L Na₂HPO₄;
30-300 mg/L NaH₂PO₄ H₂O;
0.002-0.07 mg/L SeNa₂O₃;
280-500 mg/L KCl;
40-1050 mg/L L-Asparagine H₂O;
20-1000 mg/L L-Aspartic acid;
50-1000 mg/L L-Isoleucine;
50-1200 mg/L L-Leucine;
50-500 mg/L L-Methionine;
100-1000 mg/L L-Valine;
25-1000 mg/L L-Phenylalanine;
25-430 mg/L L-Tyrosine, 2Na, 2H₂O;
100-1200 mg/L L-Lysine HCl;
50-1050 mg/L L-Threonine;
100-500 mg/L L-Histidine;
50-500 mg/L L-Serine;
2-500 mg/L L-Tryptophan;
200-5000 mg/L L-Arginine HCl;
25-250 mg/L L-Cysteine;
15-150 mg/L L-Cysteine 2HCl;
0.003-1 mg/L D-Biotin;
0.05-5 mg/L Vitamin B12;
0.05-5 mg/L Riboflavin;
0.5-20 mg/L Thiamine HCl;
0.1-7 mg/L D-calcium pantothenate;
0.5-30 mg/L Pyridoxine HCl;
1-20 mg/L Folic acid;
1-150 mg/L Choline chloride;
10-1000 mg/L Myo-inositol;
2-100 mg/L Ethanolamine HCl;
0.025-6 mg/L Putrescine 2HCl;
0.03-1 mg/L DL-α-lipoic acid;
0.01-2 mg/L Linoleic acid;
500-8000 mg/L D-Glucose;
0.001-0.02 mg/L CuSO₄ 5H₂O;
0-2 mg/L FeSO₄ 7H₂O;
0.4-2 mg/L ZnSO₄ 7H₂O;
0.00007-4.5 mg/L MnSO₄ H₂O;
5000-7500 mg/L NaCl;
0-1000 mg/L L-Proline;
0-1000 mg/L L-Glutamic acid;
0-500 mg/L Glycine;
0-1000 mg/L Sodium pyruvate;
0-20 mg/L Hypoxanthine in NaOH 1M;
0-3 mg/L Thymidine in NaOH 1M;
0-150 mg/L L-Alanine;
0-100 mg/L beta-Alanine;
0-100 mg/L L-Ornithine;
0-1000 mg/L L-Taurine;
0.9-1.1 mg/L L-α-phosphatidylcholine;
0.009-0.011 mg/L Hydrocortisone;
5300-6600 mg/L HEPES;
0-1100 mg/L Lutrol® or Pluronic® F-68;
0-7 mg/L Iron gluconate, 2H₂O;
0-200 mg/L Ferric ammonium citrate;
0.001-0.10 mg/L CoCl₂ 6H₂O;
0.001-0.005 mg/L (NH₄)₆Mo₇O₂₆ 4H₂O;
0.000025-0.0005 mg/L NiSO₄ 6H₂O;
0.02-0.4 mg/L Na₂SiO₃ 9H₂O;
0.000025-0.0005 mg/L SnCl₂ 2H₂O;
0.0001-0.0025 mg/L NH₄VO₃;
0.5-30 mg/L Nicotinamide (B3);
0.1-20 mg/L p-aminobenzoic acid;
500-650 mg/L L-Glutamine;
2000-2200 mg/L NaHCO₃;
0-110 mg/L Ferric citrate;
0-20000 mg/L Plant hydrolysates;
0-20000 mg/L Animal hydrolysates;
0-10% mg/L Serum, or any combination thereof.

Other advantages offered by the eukaryotic cell transfection system, method and media of the invention include a decrease in the amount of time and materials required for transfection of eukaryotic cells, e.g., mammalian cells, such as a reduction in the number of steps, e.g., a reduction in the amount of “hands-on” experimental time required, compared to standard and customary methods in the art, and reducing the need for specialized transfection equipment.

In certain embodiments of the systems of the invention, the transfection of eukaryotic cells is transient transfection.

In certain embodiments of the systems of the invention, the transfection of eukaryotic cells is stable transfection.

### A. Media

The cell transfection medium of the present invention requires a cryopreservant overload ratio, e.g., DMSO overload ratio, to activate the medium. Such medium for use in systems, media, and methods of the present invention augments, with a sufficient amount of cryopreservant, e.g., DMSO, to activate the medium, any medium that may be used for the growth of eukaryotic cells, e.g., mammalian primary cells, mammalian cell lines, insect cell lines, fish cell lines, or avian cell lines. In light of the inventions described herein, commercially available media that may be used in the systems, media, and methods of the present invention include, but are not limited to, all versions and varieties of ProCHO™ or PowerCHO™ Advance (Lonza), e.g., ProCHO5™, CDCHO, CD FortiCHO™, CD OptiCHO™ (ThermoFisher Scientific), Freestyle™CHO (ThermoFisher Scientific), Hycell CHO HyClone® (GE Life Sciences), DMEM-Dulbecco's Modified Eagle Medium (ThermoFisher Scientific), RPMI media (ThermoFisher Scientific, Sigma-Aldrich, Gibco, etc.), MEM-Minimum Essential Media (Gibco™), IMDM-Iscove's Modified Dulbecco's Medium (ThermoFisher Scientific), Opti-MEM™ Reduced Serum Media (Gibco™), CHO DHFR-Medium (Sigma Aldrich), EXCELL® hybridoma media (Sigma Aldrich), EXCELL® CHO media (Sigma Aldrich), EXCELL® CHOZN® media (Sigma Aldrich), EXCELL® insect cell media (Sigma Aldrich), CHO-Venture medium (Capricorn), and the like.

In certain embodiments of the invention, the medium is FlexiCHO®-CDM or variations thereof (ExcellGene SA).

In certain embodiments of the invention, the medium is FlexiHEK®-CDM or variants thereof (ExcellGene SA).

In certain embodiments of the invention, the medium is CD-CHO (ThermoFisher Scientific).

In certain embodiments of the invention, the medium is Freestyle™ Medium (ThermoFisher Scientific).

In certain embodiments of the invention, the medium is ProCHO5™ (Lonza). In certain embodiments of the invention, the medium is FlexiFly®-S2, FlexiFly®-SF9, FlexiFly®-Hi5 or variations thereof (ExcellGene SA).

In certain embodiments' of the invention, the medium may be made in a laboratory rather than purchased or modified by addition of components to a commercially available medium. In one particular embodiment, a representative selection of components, optional components, and range of amounts to use in the formulation of a medium are provided in Table 1. The person of skill in the art working with a preferred cell type or cell line would know and appreciate the components and amounts most suitable for the cell type or cell line.

TABLE 1
Exemplary media components and range of amounts suitable for use in certain
embodiments of the present invention for preparing a medium for growing
and transfecting mammalian cells (prior to activating with a cryopreservant
overload ratio, e.g., DMSO overload ratio, or pre-conditioning).

Concentration
range mg/L

| Medium component | Low | High |
|---|---|---|
| Albumin (bovine, human, etc.) | 0 | 10000 |
| Antifoam | 0 | 20000 |
| AMMONIUM METAVANADATE | 0.0001 | 0.0025 |
| AMMONIUM MOLYBDATE 4H2O | 0.001 | 0.005 |
| Animal Casein peptone | 0 | 15000 |
| Animal Peptone P6838 | 0 | 15000 |
| Animal Primatone ® HS | 0 | 15000 |
| Animal Primatone ® HS/UF | 0 | 15000 |
| Animal Primatone ® P37 | 0 | 15000 |
| Animal Primatone ® RLT | 0 | 15000 |
| Animal Primatone ® RL | 0 | 15000 |
| CADMIUM CL HEMI PENTAHYDRATE | 0 | 2 |
| CALCIUM CHLORIDE | 5 | 200 |
| CALCIUM D-PANTOTHENATE | 0.1 | 7 |
| CHOLINE CHLORIDE | 1 | 150 |
| COBALT CHLORIDE 6H2O | 0.001 | 0.1 |
| COPPER SULFATE-5H2O | 0.001 | 0.02 |
| CUPRIC/COPPER CHLORIDE-2H2O | 0 | 20 |
| D-ALPHA-TOCOPHEROL (Vit. E) | 0 | 1 |
| D-BIOTIN | 0 | 1 |
| D-GLUCOSE | 500 | 15000 |
| D-MANNITOL | 0 | 100 |
| DL-ALPHA-LIPOIC ACID | 0.02 | 1 |
| DL-ALPHA-TOCOPHEROL ACETATE | 0 | 1 |
| ETHANOLAMINE HYDROCHLORIDE | 2 | 100 |
| Fe gluconate 2 H₂0 | 0 | 7 |
| FERRIC AMMONIUM CITRATE | 0 | 100 |
| FERRIC NITRATE-9H₂O | 0 | 0.08 |
| FERROUS SULFATE 7H₂O | 0 | 2 |
| FOLIC ACID | 0 | 100 |
| GALACTOSE | 0 | 5000 |
| GLYCINE | 0 | 500 |
| HEPES, FREE ACID | 3500 | 6600 |
| HYDROCORTISONE | 0.002 | 0.011 |
| HYDROXY-L-PROLINE | 0 | 1000 |
| HYPOXANTHINE in NaOH | 0 | 20 |
| INSULIN-HUMAN RECOMBINANT | 0 | 100 |
| L-A-AMINO-N-BUTYRIC ACID | 0 | 300 |
| L-ALANINE | 0 | 150 |
| L-Alpha-phosphatidylcholin | 0.2 | 1.5 |
| L-ARGININE | 200 | 5000 |
| L-ARGININE-HCL | 200 | 5000 |
| L-ASCORBIC ACID | 0 | 1000 |
| L-ASPARAGINE | 40 | 1000 |
| L-ASPARAGINE-H2O | 40 | 1000 |
| L-ASPARTIC ACID | 20 | 1000 |
| L-CYSTEINE-HCL-H2O | 15 | 150 |
| L-CYSTINE-2HCL | 25 | 250 |
| L-GLUTAMIC ACID | 0 | 1000 |
| L-GLUTAMIC ACID MONOPOTASSIUM H₂O | 0 | 2000 |
| L-Glutamine | 250 | 1000 |
| L-GLUTATHIONE REDUCED | 0 | 1000 |
| L-HISTIDINE | 100 | 500 |
| L-HISTIDINE-HCL-H2O | 0 | 500 |
| L-ISOLEUCINE | 50 | 1000 |
| L-LEUCINE | 50 | 1500 |
| L-LYSINE-HCL | 100 | 1500 |
| L-METHIONINE | 50 | 500 |
| L-ORNITHINE HCL | 0 | 100 |
| L-PHENYLALANINE | 25 | 1000 |
| L-PROLINE | 0 | 1000 |
| L-SERINE | 50 | 500 |
| L-TAURINE | 0 | 1000 |
| L-THREONINE | 50 | 1000 |
| L-Tryptophan | 20 | 500 |
| L-TYROSINE | 25 | 800 |
| L-VALINE | 100 | 1000 |
| LINOLEIC ACID | 0.01 | 2 |
| LONG ®R³ IGF-I HUMAN | 0 | 100 |
| MAGNESIUM CHLORIDE | 15 | 70 |
| MAGNESIUM SULFATE | 20 | 100 |
| MANGANESE SULFATE H2O | 0.00005 | 4.5 |
| METHYLATED BETA-CYCLODEXTRIN | 0 | 50 |
| MYO-INOSITOL | 1 | 10000 |
| NICKEL SULFATE-6H2O | 0.000025 | 0.0005 |
| NICONTINAMIDE | 0.5 | 30 |
| OLEIC ACID | 0 | 1000 |
| PARA-AMINOBENZOIC ACID | 0.1 | 20 |
| Plant Hypep ® 1510 | 0 | 15000 |
| Plant Hypep ® 4601 | 0 | 15000 |
| Plant HYPEPR 4601N | 0 | 15000 |
| Plant Hypep ® 4605 | 0 | 15000 |
| Plant Pea peptone A282 | 0 | 15000 |
| Plant PHOSPHOLIPON ® 90G | 0 | 10000 |
| Plant Potato Peptone ET1LS | 0 | 15000 |
| Plant UltraPep ™ Soy | 0 | 15000 |
| POLOXAMER 188 (Pluronic ®, Lutrol ® F68) | 0 | 1000 |
| POTASSIUM CHLORIDE | 250 | 5000 |
| PUTRESCINE-2HCL | 0.025 | 6 |
| PYRIDOXINE-HCL | 0.5 | 30 |
| RIBOFLAVIN | 0.05 | 5 |
| SeNa₂O₃ | 0.002 | 0.07 |
| SERUM (HU, BOVINE, EQUINE, ETC.) in % | 0 | 10 |
| SODIUM CHLORIDE | 4000 | 7500 |
| SODIUM CITRATE 2H₂O | 0 | 1000 |
| SODIUM HYDROGEN CARBONATE | 1000 | 2200 |
| SODIUM METASILICATE-9H₂O | 0.02 | 0.4 |
| SODIUM PHOSPHATE DIBASIC | 30 | 100 |
| SODIUM PHOSPHATE MONOBASIC H20 | 30 | 300 |
| SODIUM PYRUVATE | 0 | 1000 |
| SODIUM SELENITE | 0 | 100 |
| STANNOUS CHLORIDE 2H₂O | 0.000025 | 0.0005 |
| STRONTIUM CHLORIDE HEXAHYDRATE | 0 | 100 |
| SUCCINIC ACID | 0 | 1000 |
| SYNTHETIC CHOLESTEROL | 0 | 30 |
| THIAMINE-HCL | 0.5 | 20 |
| Thymidine in NaOH 1M | 0 | 3 |
| TROPOLONE | 0 | 0.5 |
| TWEEN 80 | 0 | 1000 |
| VITAMIN B12 | 0.05 | 5 |
| YEAST EXTRACT, ULTRAFLTR, DRIED | 0 | 10000 |
| ZINC SULFATE-7H₂O | 0.2 | 2 |

TABLE 2
Exemplary media components and range of amounts suitable for
use in certain embodiments of the present invention for preparing
a medium for growing and transfecting mammalian cells (prior
to activating with a cryopreservant overload ratio, e.g.,
DMSO overload ratio, or pre-conditioning).

| | Concentration |
|---|---|
| Medium component | ranges mg/L |
| CaCl₂ (anhydrous) | 5-200 |
| MgCl₂ (anhydrous) | 15-70 |
| MgSO₄ (anhydrous) | 20-110 |
| Na₂HPO₄ | 30-100 |
| NaH₂PO₄ H2O | 30-300 |
| SeNa₂O₃ | 0.002-0.07 |
| Potassium chloride | 280-5000 |
| L-Asparagine H₂O | 40-1050 |
| L-Aspartic acid | 20-1000 |
| L-Isoleucine | 50-1000 |
| L-Leucine | 50-1200 |
| L-Methionine | 50-500 |
| L-Valine | 100-1000 |
| L-Phenylalanine | 25-1000 |
| L-Tyrosine, 2Na, 2H₂O | 25-430 |
| L-Lysine HCl | 100-1200 |
| L-Threonine | 50-1050 |
| L-Histidine | 100-500 |
| L-Serine | 50-500 |
| L-Tryptophan | 2-500 |
| L-Arginine HCl | 200-5000 |
| L-Cysteine | 25-250 |
| L-Cysteine 2HCl | 15-150 |
| D-Biotin | 0.003-1 |
| Vitamin B12 | 0.05-5 |
| Riboflavin | 0.05-5 |
| Thiamine HCl | 0.5-20 |
| D-calcium pantothenate | 0.1-7 |
| Pyridoxine HCl | 0.5-30 |
| Folic acid | 0.5-20 |
| Choline chloride | 1-150 |
| Myo-inositol | 10-1000 |
| Ethanolamine HCl | 2-100 |
| Putrescine 2HCl | 0.025-6 |
| DL-α-lipoic acid | 0.03-1 |
| Linoleic acid | 0.01-2 |
| D-Glucose | 500-8000 |
| CuSO₄ 5H₂O | 0.001-0.02 |
| ZnSO₄ 7H₂O | 0.4-2 |
| MnSO₄ H₂O | 0.00007-4.5 |
| NaCl | 5000-7500 |

| Optional Components | mg/liter |
|---|---|
| Fe(NO₃)₃ 9H2O | 0-0.08 |
| FeSO₄ 7H₂O | 0-2 |
| L-Proline | 0-1000 |
| L-Glutamic acid | 0-1000 |
| Glycine | 0-500 |
| Sodium pyruvate | 0-1000 |
| Hypoxanthine in NaOH 1M | 0-20 |
| Thymidine in NaOH 1M | 0-3 |
| L-Alanine | 0-150 |
| L-Ornithine | 0-100 |
| L-Taurine | 0-1000 |
| L-α-phosphatidylcholine | 0.9-1.1 |
| Hydrocortisone | 0-0.011 |
| HEPES | 0-6600 |
| Lutrol ® F-68 | 0-1100 |
| Iron gluconate, 2H₂O | 0-7 |
| Ferric ammonium citrate | 0-200 |
| CoCl₂ 6H₂0 | 0-0.10 |
| (NH₄)6Mo₇O₂₆ 4H₂O | 0-0.005 |
| NiSO₄ 6H₂O | 0-0.0005 |
| Na₂SiO₃ 9H₂O | 0-0.4 |
| SnCl₂ 2H₂O | 0-0.0005 |
| NH₄VO₃ | 0-0.0025 |
| Nicotinamide (B3) | 0-30 |
| p-aminobenzoic acid | 0-20 |
| L-Glutamine | 0-650 |
| NaHCO₃ | 0-2200 |
| Ferric citrate | 0-110 |
| Plant derived hydrolysates | 0-20000 |
| Animal derived hydrolysates | 0-20000 |
| Serum | 0-10% |

In certain embodiments of the systems of the invention, the medium (prior to activating with a cryopreservant overload ratio, e.g., DMSO overload ratio, or pre-conditioning) comprises:
5-200 mg/L CaCl2 (anhydrous);
15-70 mg/L MgCl2 (anhydrous);
0.01-0.08 mg/L Fe(NO3)3 9H2O;
20-110 mg/L MgSO4 (anhydrous);
30-100 mg/L Na2HPO4;
30-300 mg/L NaH2PO4 H2O;
0.002-0.07 mg/L SeNa2O3;
280-500 mg/L KCl;
40-1050 mg/L L-Asparagine H2O;
20-1000 mg/L L-Aspartic acid;
50-1000 mg/L L-Isoleucine;
50-1200 mg/L L-Leucine;
50-500 mg/L L-Methionine;
100-1000 mg/L L-Valine;
25-1000 mg/L L-Phenylalanine;
25-430 mg/L L-Tyrosine, 2Na, 2H2O;
100-1200 mg/L L-Lysine HCl;
50-1050 mg/L L-Threonine;
100-500 mg/L L-Histidine;
50-500 mg/L L-Serine;
2-500 mg/L L-Tryptophan;
200-5000 mg/L L-Arginine HCl;
25-250 mg/L L-Cysteine;
15-150 mg/L L-Cysteine 2HCl;
0.003-1 mg/L D-Biotin;
0.05-5 mg/L Vitamin B12;
0.05-5 mg/L Riboflavin;
0.5-20 mg/L Thiamine HCl;
0.1-7 mg/L D-calcium pantothenate;
0.5-30 mg/L Pyridoxine HCl;
1-20 mg/L Folic acid;
1-150 mg/L Choline chloride;
10-1000 mg/L Myo-inositol;
2-100 mg/L Ethanolamine HCl;
0.025-6 mg/L Putrescine 2HCl;
0.03-1 mg/L DL-α-lipoic acid;
0.01-2 mg/L Linoleic acid;
500-8000 mg/L D-Glucose;
0.001-0.02 mg/L CuSO4 5H2O;
0.05-2 mg/L FeSO4 7H2O;
0.4-2 mg/L ZnSO4 7H2O;
0.00007-4.5 mg/L MnSO4 H2O;
5000-7500 mg/L NaCl;
0-1000 mg/L L-Proline;
0-1000 mg/L L-Glutamic acid;
0-500 mg/L Glycine;
0-1000 mg/L Sodium pyruvate;
0-20 mg/L Hypoxanthine in NaOH 1M;
0-3 mg/L Thymidine in NaOH 1M;
0-150 mg/L L-Alanine;
0-100 mg/L L-Ornithine;
0-1000 mg/L L-Taurine;
0.9-1.1 mg/L L-α-phosphatidylcholine (optional);
0.009-0.011 mg/L Hydrocortisone (optional);
5300-6600 mg/L HEPES (optional);
900-1100 mg/L Lutrol® F-68 (optional);
5-7 mg/L Iron gluconate, 2H2O (optional);
0.04-200 mg/L Ferric ammonium citrate (optional);
0.001-0.10 mg/L CoCl2 6H2O (optional);
0.001-0.005 mg/L (NH4)6Mo7O26 4H2O (optional);
0.000025-0.0005 mg/L NiSO4 6H2O (optional);
0.02-0.4 mg/L Na2SiO3 9H2O (optional);
0.000025-0.0005 mg/L SnCl2 2H2O (optional);
0.0001-0.0025 mg/L NH4VO3 (optional);
0.5-30 mg/L Nicotinamide (B3) (optional);
0.1-20 mg/L p-aminobenzoic acid (optional);
500-650 mg/L L-Glutamine (optional);
2000-2200 mg/L NaHCO3 (optional);
90-110 mg/L Ferric citrate (optional);
100-20000 mg/L Plant hydrolysates (optional);
100-20000 mg/L Animal hydrolysates (optional);
0.1-10% mg/L Serum (optional); or any combination thereof.

In certain embodiment of the invention, the transfection media are designed to transfect insect cells, e.g., SF-9 cells, S2 cells, or Hi-five cells; without intending to be limiting with respect to other insect cells. As such, in certain embodiments, the systems of the invention designed to transfect insect cells comprise a cell transfection medium, e.g., Fly-M4Tx® (ExcellGene SA), further capable of supporting and facilitating insect cell growth, e.g., any of the above-referenced media, or e.g., any media according to Table 3, e.g., any media according to a combination of components according to Table 1 and Table 2.

TABLE 3
Exemplary media components and range of amounts suitable
for use in certain embodiments of the present invention
for preparing a medium for growing and transfecting insect
cells (prior to activating with a cryopreservant overload
ratio, e.g., DMSO overload ratio, or pre-conditioning).

| | Concentration | |
|---|---|---|
| | range | |
| | From | To |
| Components | (mg/L) | (mg/L) |
| Alpha-Ketoglutaric Acid | 10 | 30 |
| Beta-Alanine | 0 | 300 |
| Calcium chloride dihydrate | 200 | 800 |
| Choline chloride | 20 | 100 |
| Cobalt chloride hexahydrate | 0.05 | 0.1 |
| Cupric chloride dihydrate | 0.05 | 0.2 |
| Cyanocobalamine | 0.1 | 0.24 |
| D-Biotin | 0.05 | 0.16 |
| D-Pantothenic acid (hemicalcium) | 0.002 | 0.008 |
| D(+) Fructose | 0 | 1000 |
| D(+) Glucose | 2500 | 12500 |
| D(+) Sucrose | 0 | 2000 |
| Ferrous sulphate heptahydrate | 0 | 0.55 |
| Folic acid | 0.02 | 0.08 |
| Fumaric acid, free acid | 1 | 4.4 |
| Glycine | 100 | 300 |
| Hydroxy-L-Proline | 250 | 1000 |
| L-Arginine hydrochloride | 200 | 800 |
| L-Asparagine | 500 | 2000 |
| L-Aspartic acid | 500 | 1500 |
| L-Cystine dihydrochloride | 100 | 300 |
| L-Glutamic acid | 500 | 2000 |
| L-Histidine hydrochloride | 150 | 248 |
| L-Isoleucine | 200 | 750 |
| L-Leucine | 100 | 250 |
| L-Lysine hydrochloride | 200 | 700 |
| L-Methionine | 200 | 1000 |
| L-Phenylalanine | 200 | 1000 |
| L-Proline | 100 | 500 |
| L-Serine | 100 | 200 |
| L-Threonine | 100 | 200 |
| L-Tryptophan | 100 | 100 |
| L-Tyrosine disodium salt | 250 | 360 |
| L-Valin | 200 | 500 |
| L(−) - Malic Acid, free acid | 10 | 54 |
| Lipid mixture (L0228 Sigma) (in % of vol) | 0 | 2 |
| Magnesium sulphate anhydrous | 250 | 1000 |
| Maltose | 0 | 1000 |
| Manganese chloride | 0.005 | 0.02 |
| Molybdic acid Ammonium tetrahydrate | 0.01 | 0.04 |
| Myo-Inositol | 0.01 | 0.04 |
| Nicotine amide | 0.05 | 0.16 |
| p-Amino benzoic acid (PABA) | 0.1 | 1 |
| Plant Hypep ® 1510 | 0 | 15000 |
| Plant Hypep ® 4601 | 0 | 15000 |
| Plant HYPEP ® 4601N | 0 | 15000 |
| Plant Hypep ® 4605 | 0 | 15000 |
| Plant Pea peptone A282 | 0 | 15000 |
| Plant PHOSPHOLIPON ® 90G | 0 | 15000 |
| Plant Potato Peptone ET1LS | 0 | 15000 |
| Plant UltrPep ™Soy | 0 | 15000 |
| POLOXAMER 188 (F68, Pluronic ®, | 0 | 1000 |
| Koliphore, Pluronic ®) | | |
| Potassium chloride | 200 | 1200 |
| Pyridoxine hydrochloride | 0.01 | 0.4 |
| Riboflavin | 0.01 | 0.08 |
| Serum (fetal bovine) (in % of vol) | 0 | 5 |
| Sodium bicarbonate | 100 | 400 |
| Sodium dihydrogen phosphate | 200 | 1000 |
| Soy peptone (A3SC, Organotechnie) | 0 | 10000 |
| Succinic acid | 2.4 | 10 |
| Sucrose | 200 | 1700 |
| Thiamine hydrochloride | 0 | 0.08 |
| Yeast extract ultrafiltered | 0 | 10000 |
| Yeastolate (Difco TC) (BD) | 0 | 8000 |
| Zinc chloride | 0.01 | 0.04 |

### B. Positively Charged Polymers

In certain embodiments, the systems, media, and methods of the present invention utilize a positively charged polymer for cell transfection. The positively charged polymer may be selected from any known positively charged polymer which is suitable for pre-conditioning cell transfection medium sufficient to accommodate the systems, media, and methods of the present invention. In certain embodiments, the positively charged polymer is selected from the group consisting of any polyalkylenimine, dialkylaminoalkyl-dextran, polylysine, dendritic polylysine, hyperbranched polylysine analogues, polyarginine, PDMAEMAs (poly(2-(dimethylamino)ethyl methacrylate), polyamido amines, polyamido amine dendrimers (PAMAM), polyacylhydrazone, chitosan, cationic cellulose, cationic dextrans, spermine, dextran-spermine, spermidine, and similar polycationic substances, and derivatives or combinations thereof. Moreover, the amount of positively charged polymer suitable for use in the systems, media, and methods of the present invention is any amount of polymer sufficient to pre-condition the media that results in the transfection of purified, unencumbered exogenous nucleic acid into eukaryotic cells.

In certain embodiments of the present invention, the positively charged polymer is selected from the group consisting of polyalkylenimine, dialkylaminoalkyl-dextran, polylysine, dendritic polylysine, hyperbranched polylysine analogues and derivatives thereof, any naturally occurring polycations (such as Chitosans, cationic cellulose, cationic dextrans, spermines, dextran-spermine, and derivatives thereof), and any derivatives or combinations thereof.

In certain embodiments of the invention, the positively-charge polymer is polyethylenimine. In certain embodiments, the polyethylenimine is linear, branched or hyperbranched. In particular embodiments, the polyethylenimine is linear. In one embodiment, the positively charged polymer used to pre-condition the medium is added at a final concentration of 1 mg/L medium to 50 mg/L medium.

In certain embodiments of the invention, the polyethylenimine is at least 10 KDa in molecular weight. In certain embodiments, the polyethylenimine is 25 KDa in molecular weight. In certain alternative embodiments, the polyethylenimine is 10 KDa in molecular weight. In certain embodiments the polyethylenimine is a mixture of two or more PEI of different average molecular weights, e.g., a mixture of 10 KDa molecular weight and 25 KDa molecular weight PEI, a mixture of 25 KDa molecular weight and 40 KDa molecular weight PEI, a mixture of 10 KDa molecular weight and 40 KDa molecular weight PEI, or a mixture of 10 KDa molecular weight, 25 KDa molecular weight and 40 KDa molecular weight PEI,

In one embodiment, the PEI suitable for use in the practice of the system and method of the present invention is linear. In certain embodiments, the linear PEI is deacylated. In certain embodiment, the linear PEI has an average molecular weight of at least 800 Daltons, e.g., 1 to 5 KDa, e.g., 5 to 10 KDa, e.g., 10 to 15 KDa, e.g., 15 to 20 KDa, e.g., 20 to 25 KDa, e.g., 25 to 30 KDa, e.g., 30 to 40 KDa, e.g., 40 to 50 KDa, or more. In certain embodiments, the linear PEI has an average molecular weight of at least 5 KDa, e.g., 5 to 10 KDa, e.g., 10 to 15 KDa, e.g., 15 to 20 KDa, e.g., 20 to 25 KDa, e.g., 25 to 30 KDa, e.g., 30 to 40 KDa, e.g., 40 to 50 KDa, or more. In certain embodiments, the linear PEI has an average molecular weight of at least 10 KDa, e.g., 10 to 15 KDa, e.g., 15 to 20 KDa, e.g., 20 to 25 KDa, e.g., 25 to 30 KDa, e.g., 30 to 40 KDa, e.g., 40 to 50 KDa, or more. In certain embodiments, the linear PEI has an average molecular weight of at least 20 KDa, e.g., 20 to 25 KDa, e.g., 25 to 30 KDa, e.g., 30 to 40 KDa, e.g., 40 to 50 KDa, e.g., 50 to 100 KDa, or more. In one embodiment, the linear PEI has an average molecular weight of about 10 KDa. In another embodiment, the linear PEI has an average molecular weight of about 25 KDa.

In one embodiment, the PEI is linear 10 KDa PEI added at a final concentration of 1 mg/L medium to 50 mg/L medium. In another embodiment, the linear 10 KDa PEI is added at a final concentration of 2 mg/L medium to 40 mg/L medium. In another embodiment, the linear 10 KDa PEI is added at a final concentration of 3 mg/L medium to 30 mg/L medium. In yet another embodiment, the linear 10 KDa PEI is added at a final concentration of 4 mg/L medium to 20 mg/L medium. In another embodiment, the linear 10 KDa PEI is added at a final concentration of 5 mg/L medium to 10 mg/L medium. In another embodiment, the linear 10 KDa PEI is added at a final concentration of 6 mg/L medium to 10 mg/L medium. In another embodiment, the linear 10 KDa PEI is added at a final concentration of 7 mg/L medium to 10 mg/L medium. In another embodiment, the linear 10 KDa PEI is added at a final concentration of 8 mg/L medium to 10 mg/L medium. In another embodiment, the linear 10 KDa PEI is added at a final concentration of 8 mg/L medium to 9 mg/L medium. In another embodiment, the linear 10 KDa PEI is added at a final concentration of 9 mg/L medium to 10 mg/L medium.

In another embodiment, the PEI is linear 25 KDa PEI added at a final concentration of 1 mg/L medium to 50 mg/L medium. In another embodiment, the linear 25 KDa PEI is added at a final concentration of 2 mg/L medium to 40 mg/L medium. In another embodiment, the linear 25 KDa PEI is added at a final concentration of 3 mg/L medium to 30 mg/L medium. In yet another embodiment, the linear 25 KDa PEI is added at a final concentration of 4 mg/L medium to 20 mg/L medium. In another embodiment, the linear 25 KDa PEI is added at a final concentration of 5 mg/L medium to 10 mg/L medium. In another embodiment, the linear 25 KDa PEI is added at a final concentration of 6 mg/L medium to 10 mg/L medium. In another embodiment, the linear 25 KDa PEI is added at a final concentration of 7 mg/L medium to 10 mg/L medium. In another embodiment, the linear 25 KDa PEI is added at a final concentration of 8 mg/L medium to 10 mg/L medium. In another embodiment, the linear 25 KDa PEI is added at a final concentration of 8 mg/L medium to 9 mg/L medium. In another embodiment, the linear 25 KDa PEI is added at a final concentration of 9 mg/L medium to 10 mg/L medium.

### C. Nucleic Acid

In one embodiment, the nucleic acid suitable for transfection of eukaryotic cells (e.g., mammalian cells such as human cells or animal cells, insect cells, fish cells, plant cells, fungal cells, and avian cells) is any type or form of natural or synthetic polymers of deoxyribonucleic acid (DNA) or ribonucleic acid (RNA), or biopolymers comprising a mix of both deoxyribonucleotides and ribonucleotides (DNA-RNA hybrids), peptide nucleic acid (PNA), or locked nucleic acid (LNA), and the like. In certain embodiments, DNA biopolymers include, but are not limited to, circular DNA forms, e.g., plasmids, linear DNA, e.g., amplified DNA products, e.g., DNA viral genomes, and the like. In a particular embodiment, the DNA biopolymer transfected into a cell using the system and method of the invention is plasmid DNA. In certain embodiments, RNA biopolymers include, but are not limited to, messenger RNA, e.g., mRNA, transfer RNA, e.g., tRNA, ribosomal RNA, e.g., rRNA, heterologous nuclear RNA, e.g., hn-RNA, small nuclear RNA, e.g., sn-RNA, small nucleolar RNA, any type of RNA capable of interfering with the transcription or translation of a gene, such as small interfering RNA, e.g., siRNA, micro RNA, e.g., miRNA, e.g., long-noncoding RNA, e.g., guide RNA, e.g., CRISPR RNA, e.g., short hairpin RNA, e.g., doggy bone DNA, e.g., anti-sense RNA, e.g., enhancer RNA, RNA viral genomes, and the like.

In certain embodiments of the invention, the nucleic acid is selected from the group consisting of DNA, RNA, PNA, LNA, and any combination thereof.

In certain embodiments of the invention, the nucleic acid is DNA. In a particular embodiment, the DNA is circular. In a particular embodiment, the DNA is supercoiled DNA. In an alternative particular embodiment, the DNA is linear. In another particular embodiment the nucleic acid is a mixture of linear and/or circular DNA and/or supercoiled DNA. In yet another embodiment, the nucleic acid further comprises sheared DNA.

In certain embodiments of the invention, the nucleic acid is RNA. In certain embodiments, the RNA is selected from the group consisting of mRNA, rRNA, hn-RNA, mi-RNA, sn-RNA, small nucleolar RNA, long-noncoding RNA, guide RNA, micro RNA, CRISPR RNA, anti-sense RNA, small interfering RNA, short hairpin RNA, enhancer RNA, any RNA capable of interfering with gene expression, and/or any combination thereof.

In certain embodiments of the invention, the nucleic acid has a nucleotide sequence that is 0% to 100% identical to, or complementary to, a DNA or RNA sequence already existing in the recipient cell, e.g., a eukaryotic cell, e.g., a mammalian cell (e.g., a human cell or an animal cell), an insect cell, a fish cell, a plant cell, a fungal cell, or an avian cell. In one embodiment, the sequence of exogenous nucleic acid is not identical to, e.g., may differ in whole or in part, from a sequence already existing in the recipient cell, for example, the exogenous nucleic acid may be different from a DNA or RNA sequence already existing in the recipient cell, for example, 100% different (0% identical), 99% to 90% different (1% to 10% identical), 89% to 80% different (11% to 20% identical), 79% to 70% different (21% to 30% identical), 69% to 60% different (31% to 40% identical), 59% to 50% different (41% to 50% identical), 49% to 40% different (51% to 60% identical), 39% to 30% different (61% to 70% identical), 29% to 20% different (71% to 80% identical), 19% to 10% different (81% to 90% identical), 9% to 1% different (91% to 99% identical), 0.5% different (99.5% identical), 0.1% different (99.9% identical), or even 0.01% different (99.99% identical).

In certain embodiments of the systems, media, and methods of the invention, the purified, unencumbered exogenous nucleic acid is free from one or more of the components selected from the group consisting of proteins, cationic lipids, cationic polymers, liposome forming components, calcium phosphates, calcium chloride, nano-particles, metals, polymeric gene carriers, dendrimers, cyclodextrins, and any combination thereof. In particular embodiments, the purified, unencumbered exogenous nucleic acid is free from all components selected from the group consisting of proteins, cationic lipids, cationic polymers, liposome forming components, calcium phosphates, calcium chloride, nano-particles, metals, polymeric gene carriers, dendrimers, and cyclodextrins. In certain embodiments, the purified, unencumbered exogenous nucleic acids may exist in an essentially solvent-free form, e.g., as a pellet of material, or dissolved in an aqueous solution such as purified water, or TE buffer, e.g., 10 mM Tris 1 mM EDTA buffer, and the like.

### D. Cells

The eukaryotic cells suitable for use with the systems, media, and methods of the present invention are any eukaryotic cells, e.g., mammalian cells (e.g., human cells), animal cells (e.g., fish cells, metazoan cells, or avian cells), insect cells, plant cells, or fungal cells, capable of being transfected with exogenous nucleic acid.

In certain embodiments of the present invention, the eukaryotic cell concentration is at least 40×10⁶ cells/mL, e.g., at least 60×10⁶ cells/mL. In certain embodiments, range from about 40×10⁶ cells/mL to about 300×10⁶ cells/mL, e.g., from about 40×10⁶ cells/mL to about 100×10⁶ cells/mL, e.g., from about 60×10⁶ cells/mL to about 100×10⁶ cells/mL.

In certain embodiments of the present invention, the eukaryotic cell is selected from the group consisting of a mammalian cell, a fish cell, an insect cell, an avian cell, metazoan cell, and any combination thereof. In certain embodiments, the eukaryotic cell is a mammalian cell. In certain embodiments, mammalian cell is selected from the group consisting of primary cells isolated from a mammal or are cells that have been established as immortalized cell lines. In particular embodiments, the mammalian cell is a CHO cell. In specific embodiments, the CHO cell is selected from the group consisting of a CHO K1-derived cell, a CHO S-derived cell, CHO DG44-derived cell, CHO DUKX B11-derived cell, a CHOExpress® cell, a CHO2Express® cell, a CHOice® cell, and a CHO4Tx® cell.

In certain embodiments of the systems, media, and methods of the invention, eukaryotic cells are mammalian cells, e.g., cultivated mammalian cells. In certain embodiments, the mammalian cells are selected from the group consisting of primary cells isolated from an animal, e.g., from a mammal, e.g., from a hamster (e.g., Chinese hamster ovary (CHO) cells); from a human (e.g., human embryonic kidney cells, human cervical cancer cells, human prostate cancer cells, human adrenocortical cancer cells, human chronic myelogenous leukemia cells, human prostate cancer cells, human breast cancer cells, human bone cancer cells, human neuroblastoma cells, human acute myeloid leukemia cells, or human glioblastoma cells); from a monkey (e.g. monkey kidney epithelial cells); from a mouse (e.g., mouse embryonic calvarium cells); from a rat (e.g., rat pituitary tumor cells or rat pheochromocytoma cells); and from a dog (e.g., dog kidney epithelial cells). In particular embodiments, the mammalian cell is a CHO cell. In specific embodiments, the CHO cell is selected from the group consisting of a CHO-ori derived cell, CHO K1-derived cell, a CHO S-derived cell, CHO DG44-derived cell, CHO DUKX B11-derived cell, a CHOExpress® cell, a CHO2Express® cell, a CHOice® cell, and a CHO4Tx® cell.

In certain embodiments of the invention, the mammalian cell lines suitable for use in practicing the present invention include, but are not limited to any type or species of human embryonic kidney (HEK293) cells (e.g., HEKExpress®, HEK2Express®, 4Hard2Express® (ExcellGene cell lines)); human cervical cancer cells, e.g., Hela cells; human prostate cancer cells, e.g., DU145; human adrenocortical cancer cells, e.g., H295R; human chronic myelogenous leukemia cells, e.g., KBM-7 cells; human prostate cancer cells, e.g., LNCaP cells, e.g., PC3 cells; human breast cancer cells, e.g., MCF-7 cells, e.g., MDA-MB-468 cells, e.g., T-47D cells; human bone cancer cells, e.g., SaOS-2 cells; human neuroblastoma cells, e.g., SH-SY5Y; human acute myeloid leukemia cells, e.g., THP-1 cells; human glioblastoma cells, e.g., U87; monkey kidney epithelial cells, e.g., Vero cells; mouse embryonic calvarium cells, e.g., MC3T3 cells; rat pituitary tumor cells, e.g., GH3; rat pheochromocytoma cells, e.g., PC12 cells; dog kidney epithelial cells, e.g., MDCK cells; and the like.

In certain embodiments of the invention, the mammalian cells are primary mammalian cells, such as but not limited to, isolated human lymphocytes, peripheral blood mononuclear cells, tumor-infiltrating T-lymphocytes, and the like.

In certain embodiments of the invention, the systems, media, and methods of the present invention may be practiced utilizing non-mammalian, eukaryotic cell lines. In one embodiment, the non-mammalian, eukaryotic cells lines suitable for use in practicing the present invention include cells or cell lines derived from insects, fish or avian species. In certain embodiments, insect cell lines suitable for use in practicing the present invention include, but are not limited to, all species of cells from Drosophila melanogaster, e.g., Schneider 2 cells; Spodoptera frugiperda, e.g., Sf9 cells, e.g., Sf21 cells; Trichoplusia ni, e.g., High Five cells, and the like. In certain embodiments, fish cell lines suitable for use in practicing the present invention include, but are not limited to, all species of Zebra fish fin fibroblast cells, e.g., AB9 cells. In certain embodiments, avian cell lines suitable for use in practicing the present invention include, but are not limited to, chick embryo fibroblasts (CEF); quail fibroblast cells, e.g., QT-6 cells; duck embryo cells (DE); quail fibrosarcoma cells (QF); goose embryonic epithelial cell line (GEE), chicken fibroblast cell lines, e.g., DF-1; and the like.

In one embodiment, the eukaryotic cell transfection system, method and media is used to transiently transfect the recipient cells. In another embodiment, the eukaryotic cell transfection system, method and media is used to stably transfect the recipient cells. In light of the disclosure of the present invention, the skilled artisan would know and appreciate the adaptations necessary to transiently or stably transfect a desired recipient cell.

### III. Methods of the Invention

The activated frozen cell transfection systems of the present invention, which are capable of mediating transfection of a nucleic acid into a eukaryotic cell immediately upon thaw and without a pre-culture step may be utilized in methods of, as described herein.

As such, one embodiment of the present invention provides a method of transfecting eukaryotic cells comprising:
obtaining an activated frozen cell transfection system (AFCT-system) of the present invention;
thawing the activated frozen cell transfection system;
combining the thawed activated frozen cell transfection system with exogenous nucleic acid without a pre-culture step,
   such that said exogenous nucleic acid is transfected into said eukaryotic cells immediately upon thaw and without a pre-culture step. In certain embodiments, additional cell transfection medium is added to the thawed activated frozen cell transfection system. In certain embodiments, the supernatant is harvested after a given time, e.g., 7-10 days, to collect transfection product.

In certain embodiments of the present invention, the nucleic acid is a purified, unencumbered exogenous nucleic acid.

In certain embodiments of the present invention, the activated frozen cell transfection system is derived from the step of freezing an activation medium of the present invention, e.g., containing a given number of cells per mL.

In certain embodiments of the present invention, the transfection is performed at small scale, e.g., 1 L or less.

In certain embodiments of the present invention, the transfection is performed at large scale, e.g., greater than 1 L.

In certain embodiments of the present invention, the transfection is performed with reduced nucleic acid, e.g., ranging from 25% to 99% of normal load, e.g., 25% of normal load, e.g., corresponding to 1.5 μg of DNA for a 10 mL transfection, instead of 6 μg.

Another embodiment of the present invention provides a method of preparing an activated frozen cell transfection system comprising:
obtaining an activation medium of the present invention;
freezing the cell transfection medium combined with the eukaryotic cell, e.g., at −80° C.

In certain embodiments of the present invention, the eukaryotic cell is grown in a culture medium to sufficient volume and then harvested (e.g., by centrifugation) and suspended in the cell transfection medium with the cryopreservant overload ratio, e.g., DMSO overload ratio (e.g., at least 40×10⁶ cells/mL, e.g., at least 60×10⁶ cells/mL).

### IV. Activated Frozen Cell Transfection Kits of the Invention

The systems of the present invention may further be packaged as a kit for use in activated frozen cell transfection of eukaryotic cells. As such, another embodiment of the present invention provides a kit for transfection of eukaryotic cells comprising:
an activated frozen cell transfection system (AFCT-system) of the present invention, e.g., wherein said eukaryotic cells are selected from the group consisting of mammalian cells, insect cells, fish cells and avian cells; and
the nucleic acid is a DNA vector compatible with said eukaryotic cells.

In certain embodiments of the kits of the present invention, eukaryotic cells are mammalian cells. In certain embodiments, the mammalian cells are selected from the group consisting of primary cells isolated from an animal, from a hamster, e.g., Chinese hamster ovary (CHO) cells, from a human, e.g., human embryonic kidney cells, human cervical cancer cells, human prostate cancer cells, human adrenocortical cancer cells, human chronic myelogenous leukemia cells, human prostate cancer cells, human breast cancer cells, human bone cancer cells, human neuroblastoma cells, human acute myeloid leukemia cells, or human glioblastoma cells, from a monkey, e.g. monkey kidney epithelial cells, from a mouse, e.g., mouse embryonic calvarium cells, from a rat, e.g., rat pituitary tumor cells or rat pheochromocytoma cells, and from a dog, e.g., dog kidney epithelial cells. In particular embodiments, the mammalian cell is a CHO cell. In specific embodiments, the CHO cell is selected from the group consisting of a CHO K1-derived cell, a CHO S-derived cell, CHO DG44-derived cell, CHO DUKX B11-derived cell, a CHOExpress® cell, a CHO2Express® cell, a CHOice® cell, and a CHO4Tx® cell.

### EXEMPLIFICATIONS

In certain embodiments, transfection of eukaryotic cells, e.g., mammalian cells, e.g., CHO cell species, utilizing the system, method and media of the present invention may be carried out according to the following procedures. One skilled in the art, in light of the presently disclosed invention, would know that procedures similar to those described herein may be used to achieve transfection of eukaryotic cells (e.g., mammalian cells, e.g., human cells or animal cells, e.g., CHO cell species) using a variety of nucleic acids (e.g., DNA, e.g., plasmid DNA), and utilizing the systems, method and pre-conditioned media described herein.

### General Experiment

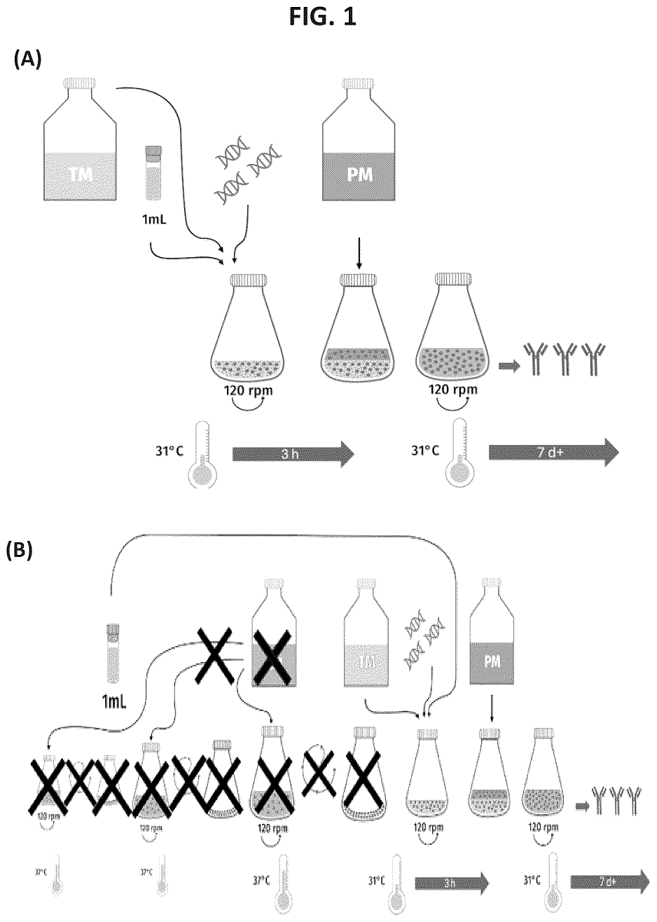

FIG. 1 is a general demonstration of the methods of transfection of the present invention utilizing an activated frozen cell transfection system (AFCT-system) of the invention. Step 1 depicts thawing and transfection without pre-culturing or specialized equipment. Step 2 shows the simplified production and collection of product of transient gene expression (TGE).

Using this general experiment framework, the following experiments will demonstrate transfection of a nucleic acid into a eukaryotic cell using an activated frozen cell transfection system of the present invention. The first step comprises thawing a frozen cell transfection medium with a DMSO overload ratio (e.g., at least 10% DMSO), cell-supplemented with eukaryotic cells. The second step comprises demonstrating the transfection of these eukaryotic cells directly upon thawing and combination with exogenous nucleic acid without a pre-culture step. Such examples will demonstrate exemplification of cells, concentrations, efficiencies transfection volumes, and stability.

The following abbreviations shall be used in the experimentals and Figures:
TS=TubeSpin® bioreactors 50 mL-size (centrifuge tube with vented cap) Working volume: 5 to 20 mL volume of transfection reaction
MTS=MaxiTubeSpin, is used for transfections of up to 300 MI, also indicated as TubeSpin bioreactor 600
Tfx=Transfection
D1, D3, D7, D10, D14=Number of days after transfection
TM=CHO4Tx® Transfection medium (part of the CHO4Tx® transfection kit)
FTM=Freezing™ medium, i.e., the activation medium of the present invention
CM=Culture medium for growth and expanding volumes of culture
PM=Production medium
VCD=Viable Cell Density
TGE=Transient Gene Expression
Mio=millions
GFP=Green Fluorescent Protein

Further, FT-CHO4Tx® cells shall be used in the following examples and are provided in an activation medium of the present invention, allowing freezing and subsequent transfection without the need for specialized equipment of pre-culturing. In particular, FT-CHO4Tx® cells have been derived from CHO4Tx® cells, but have been generated by increasing DMSO to 10% (over 4× known concentrations). These cells frozen in FTM can be transfected right out of the freezer, no need for medium exchange or pre-culture.

Working with an activated frozen eukaryotic Cell transfection (AFCT) system and related methods of the present invention can be done with several types of non-instrumented containers and orbital shaking technology, including Erlenmeyer-type cell culture flasks, which are widely used in the industry for simple cell culture of suspension cells.

In the examples noted below, the experiments were based on frozen cells in cryovials of 1 mL (Corning cryogenic vial CLS431417, or similar). Cells from these vials are used for 10 mL scale of transfection/production in 50 ml TubeSpin® bioreactors (TPP, Birsfelden, Switzerland). The standard condition for 10 mL scale transfections are below:

### Transfection in 50 mL TubeSpin® Bioreactors.

A cryovial with 60 million cells/mL with 1 mL of frozen cell suspension is rapidly thawed and the entire volume is transferred into a 50 mL TubeSpin bioreactor that has been pre-filled with 4 mL of Transfection Medium (FTM). 60 μL of a DNA-solution, at about 1 mg/mL is added immediately, or could have been added to the empty TubeSpin bioreactor before addition of Transfection Medium. The TubeSpin bioreactor, with cells, transfection medium and DNA is transferred into a shaker incubator, 31° C. and set to a shaking speed of 180 rpm (Kühner Shaker Incubator ISF-X), with a displacement radius of 50 mm, for 3 hours.

Subsequently, 5 mL of Production Medium (PM) is added to the TubeSpin bioreactor and the production is allowed to continue for 7 or 14 days, at 31° C. According to this standard method, the cell density under production conditions is about 6 million cells/mL (+/−20%) and the DNA concentration is 6 μg/mL (+/−10%) (relative to the total production volume). When DNA quantity is reduced by 50% or 75%, it will result in 3 μg/mL or 1.5 μg/mL. When the cell number in a vial is modified to a lower (40 million cells/mL) or higher cell densities than 60 million cells/mL while still containing only 1 mL, the transfection and production volume would be adjusted to a smaller or larger volume, in order to maintain the ratios of activation medium (FTM) and cells. Efficiency of transfection is typically assessed by product concentrations of an antibody-like molecule (μg/mL) at the end of a 7 or 14 day production phase. A product named A3 is used in most cases and the expression vector is named pA3. Some transfections were done with an expression vector for Green Fluorescent Protein (pGFP). In this case the transfection efficiency is verified by measuring the percentage of cells showing green fluorescence.

Scale-up experiments with transfections of the AFCT-system were executed according to the conditions as indicated for the 10 mL scale transfections (60 million cells/mL and 6 μg/mL of DNA).

### Example 1

### Transfections with FT-CHO4Tx™ Cells in FTM

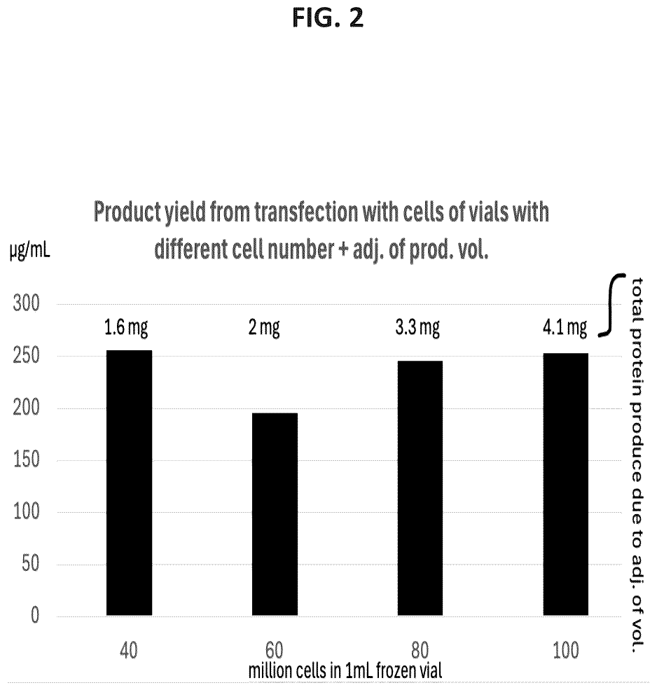

CHO4Tx® cells were harvested on day 3, centrifuged and taken up in activation medium (FTM). CHO4Tx® cells were then frozen in FTM at different cell densities. The cells were then thawed, followed by transfection (TGE). FIG. 2 depicts the results in terms of production yields (μg/mL) from FT-CHO4Tx™ cells, frozen in FTM with different cell densities and with the corresponding adjustments of the final transfection/production volume.

### Conclusions

FT-CHO4Tx™ cells can be frozen in FTM and are readily transfectable, immediately after thaw. While varying cell densities during freezing has little effect on product concentration at harvest, due to the adjustment of the production volume, significantly more product can be harvested in transfections executed with frozen cells at a higher concentration in a vial.

### Example 2

### Transfections with FT-CHO4Tx™ Cells Under Reduction of DNA

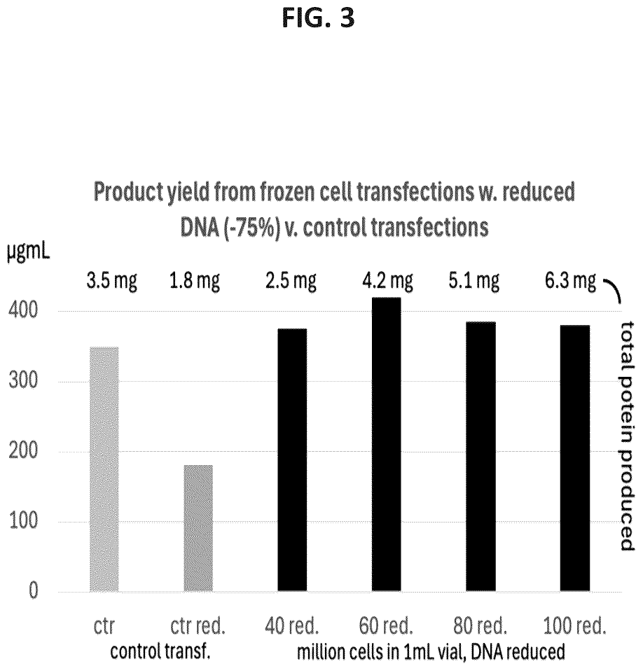

CHO4Tx® cells were harvested on day 3, centrifuged and taken up in activation medium (FTM). CHO4Tx® cells were then frozen in FTM at different cell densities. The cells were then thawed, followed by transfection (TGE) under reduction by 75% of DNA and compared to standard TGE with pre-cultured cells: FIG. 3 depicts results in terms of product yields from transfections under reduction of DNA by 75% while also modifying cell number in frozen vials (black) and adjusting the transfected volume for production. Control transfections with non-frozen cells (grey).

### Conclusions

As seen before (FIG. 2) with the standard quantity of DNA, FT-CHO4Tx™ cells performed well under reduced DNA. In addition, modifying the cell number in a vial, allows one to modify the production volume and can deliver significant increases in total protein produced.

### Example 3

### Transfections with FT-CHO4Tx™ Cells for Larger Scale Operation

CHO4Tx® cells were scaled-up to grow in 2 MTS bottles (TubeSpin® Bioreactor 600), each with 250 ml culture and at an approximate cell density of 4 mio cells/ml (day 3). The cell suspensions of the two bottles were centrifuged and the combined pellets were rapidly taken up into about 35 mL of FTM, resulting in a cell density of about 60 mio cells/mL. This volume was then transferred into a 30 mL CryoVault container (Meissner Art. no 91R71T83, HDPE). This container was frozen at −80° C. for at least 3 days before use. The 30 mL CryoVault container was thawed and the entire volume was transferred rapidly into one MTS. This MTS had just before been provided with 3 mg (about 3 mL) of DNA-vector solution and 120 mL of FTM (Transfection Medium). The bottle was then transferred into an incubator shaker for 3 hours at 31° C. Subsequently, 150 mL of Production Medium (PM) was added to obtain the final volume of 300 mL and incubation continued at 31° C. for 7 days.

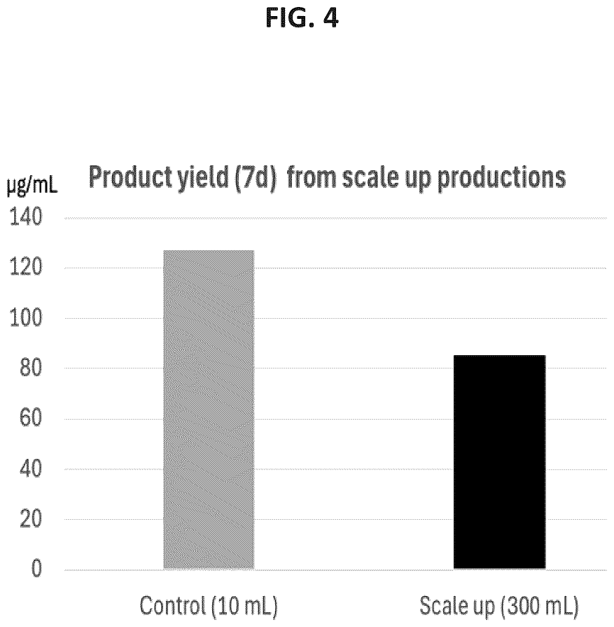

FIG. 4 depicts results in product yield upon scale-up of the method to the 300 mL scale in TubeSpin® bioreactor 600 (TPP Birsfelden, Switzerland). A control transfection is from non-frozen cells at 10 mL scale in 50 mL TubeSpin bioreactor. One 30 mL Cyrovault provides cells for one transfection at a volume of 300 mL.

### Conclusions

The protein production at the larger scale in comparison to a control transfection (day 7) was satisfying, while not fully reaching the yield of the control transfection. This might be caused by the modified handling of cells due to the larger volume needed.

### Example 4

### Stability Under Freezing Conditions Over Time of FT-CHO4Tx™ Cells

For the study of long-term storage of cells, examples of two experiments are presented: A) 1 month storage experiment was executed with frozen cells at 60 mio cells/mL and 120 mio cells/mL. Also, 75% reduction of DNA was implemented. All experiments were done at the 10 mL scale of production. The assessment of the transient transfection was done by measuring product yield in the supernatant (μg/mL). B) 6 months storage of cells was tested using a vector for GFP expression. In this case the % of green cells was determined on day 3.

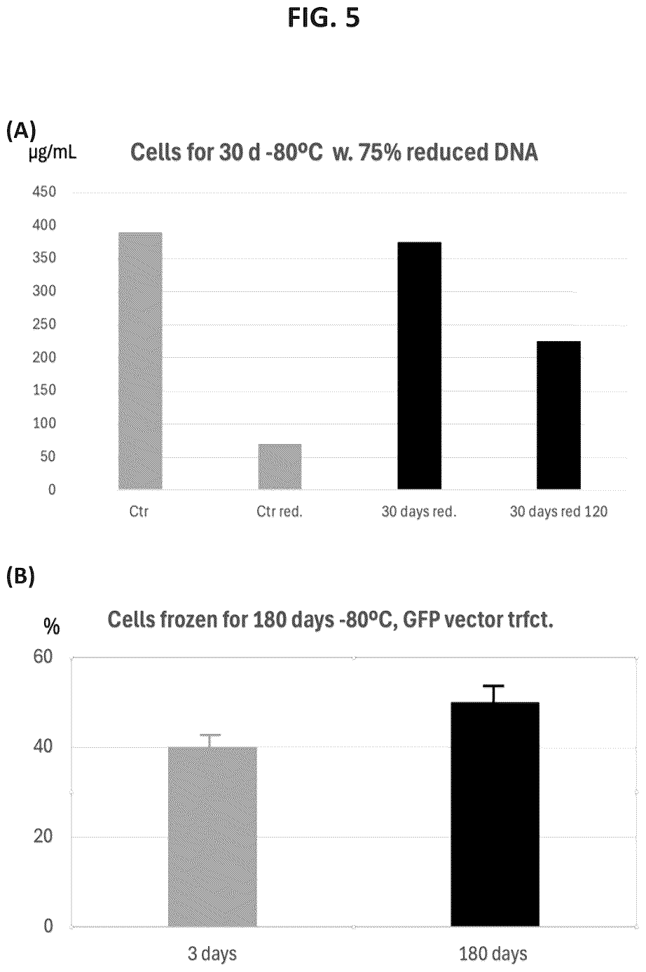

FIG. 5 depicts the storage stabilities of frozen cells at −80° C. for transfection after thaw. All transfections at 10 mL scale. A) 30 days: Transfections were executed with a reduction of DNA by 75% except in one control transfection. Productivity after 14 days in protein concentration in the supernatant. B) 180 days: Transfection efficiency as measured by GFP expression, day 3. Transfections were done as triplicates.

### Conclusions

FT-CHO4Tx cells are stable when frozen over extended periods of time for transfection after thaw.

### INCORPORATION BY REFERENCE

The entire contents of all patents, published patent applications and other references cited herein are hereby expressly incorporated herein in their entireties by reference.

### EQUIVALENTS

Detailed embodiments of the present invention are disclosed here; however, it is to be understood that the disclosed embodiments are merely illustrative of the invention that may be embodied in various forms. In addition, each of the examples given in connection with the various embodiments of the invention is intended to be illustrative, and not restrictive.

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

What is claimed is:

1. An activated frozen cell transfection system (AFCT-system) that is configured for transfection of a nucleic acid into a eukaryotic cell immediately upon thaw and without a pre-culture step comprising:
a cell transfection medium with a cryopreservant overload ratio; and
a eukaryotic cell for combination with said cell transfection medium to produce cell-supplemented cell transfection medium,
wherein the cell transfection medium combined with the eukaryotic cell are frozen.

2. The activated frozen cell transfection system of claim 1, wherein the nucleic acid is purified, unencumbered exogenous nucleic acid, and wherein the system is pre-conditioned with a positively charged polymer that is not associated with nucleic acid and is capable of mediating transfection of the purified, unencumbered exogenous nucleic acid into the eukaryotic cell.

3. The activated frozen cell transfection system of claim 1, wherein said cell transfection medium comprises:
5-200 mg/L CaCl₂) (anhydrous);
15-70 mg/L MgCl₂ (anhydrous);
0-0.08 mg/L Fe(NO₃)₃ 9H₂O;
20-110 mg/L MgSO₄ (anhydrous);
30-100 mg/L Na₂HPO₄;
30-300 mg/L NaH₂PO₄ H₂O;
0.002-0.07 mg/L SeNa₂O₃;
280-500 mg/L KCl;
40-1050 mg/L L-Asparagine H₂O;
20-1000 mg/L L-Aspartic acid;
50-1000 mg/L L-Isoleucine;
50-1200 mg/L L-Leucine;
50-500 mg/L L-Methionine;
100-1000 mg/L L-Valine;
25-1000 mg/L L-Phenylalanine;
25-430 mg/L L-Tyrosine, 2Na, 2H₂O;
100-1200 mg/L L-Lysine HCl;
50-1050 mg/L L-Threonine;
100-500 mg/L L-Histidine;
50-500 mg/L L-Serine;
2-500 mg/L L-Tryptophan;
200-5000 mg/L L-Arginine HCl;
25-250 mg/L L-Cysteine;
15-150 mg/L L-Cysteine 2HCl;
0.003-1 mg/L D-Biotin;
0.05-5 mg/L Vitamin B12;
0.05-5 mg/L Riboflavin;
0.5-20 mg/L Thiamine HCl;
0.1-7 mg/L D-calcium pantothenate;
0.5-30 mg/L Pyridoxine HCl;
1-20 mg/L Folic acid;
1-150 mg/L Choline chloride;
10-1000 mg/L Myo-inositol;
2-100 mg/L Ethanolamine HCl;
0.025-6 mg/L Putrescine 2HCl;
0.03-1 mg/L DL-α-lipoic acid;
0.01-2 mg/L Linoleic acid;
500-8000 mg/L D-Glucose;
0.001-0.02 mg/L CuSO₄ 5H₂O;
0-2 mg/L FeSO₄ 7H₂O;
0.4-2 mg/L ZnSO₄ 7H₂O;
0.00007-4.5 mg/L MnSO₄ H₂O;
5000-7500 mg/L NaCl;
0-1000 mg/L L-Proline;
0-1000 mg/L L-Glutamic acid;
0-500 mg/L Glycine;
0-1000 mg/L Sodium pyruvate;
0-20 mg/L Hypoxanthine in NaOH 1M;
0-3 mg/L Thymidine in NaOH 1M;
0-150 mg/L L-Alanine;
0-100 mg/L beta-Alanine;
0-100 mg/L L-Ornithine;
0-1000 mg/L L-Taurine;
0.9-1.1 mg/L L-α-phosphatidylcholine;
0.009-0.011 mg/L Hydrocortisone;
5300-6600 mg/L HEPES;
0-1100 mg/L Lutrol® or Pluronic® F-68;
0-7 mg/L Iron gluconate, 2H₂O;
0-200 mg/L Ferric ammonium citrate;
0.001-0.10 mg/L CoCl₂ 6H₂O;
0.001-0.005 mg/L (NH₄)₆Mo₇O₂₆ 4H₂O;
0.000025-0.0005 mg/L NiSO₄ 6H₂O;
0.02-0.4 mg/L Na₂SiO₃ 9H₂O;
0.000025-0.0005 mg/L SnCl₂ 2H₂O;
0.0001-0.0025 mg/L NH₄VO₃;
0.5-30 mg/L Nicotinamide (B3);
0.1-20 mg/L p-aminobenzoic acid;
500-650 mg/L L-Glutamine;
2000-2200 mg/L NaHCO₃;
0-110 mg/L Ferric citrate;
0-20000 mg/L Plant hydrolysates;
0-20000 mg/L Animal hydrolysates;
0-10% mg/L Serum, or any combination thereof.

4. The activated frozen cell transfection system of claim 2, wherein said positively-charge polymer is polyethylenimine.

5. The activated frozen cell transfection system of claim 1, wherein said eukaryotic cell is selected from the group consisting of a mammalian cell, a fish cell, an insect cell, an avian cell, metazoan cell, and any combination thereof.

6. The activated frozen cell transfection system of claim 5, wherein said eukaryotic cell is a mammalian cell.

7. The activated frozen cell transfection system according to claim 6, wherein said mammalian cell is selected from the group consisting of primary cells isolated from a mammal or are cells that have been established as immortalized cell lines.

8. The activated frozen cell transfection system of claim 6, wherein said mammalian cell is a CHO cell.

9. The activated frozen cell transfection system of claim 8, wherein said CHO cell is selected from the group consisting of a CHO K1-derived cell, a CHO S-derived cell, CHO DG44-derived cell, CHO DUKX B11-derived cell, a CHOExpress® cell, a CHO2Express® cell, a CHOice® cell, and a CHO4Tx® cell.

10. The activated frozen cell transfection system of any of claim 1, wherein said nucleic acid is selected from the group consisting of DNA, RNA, PNA, LNA, and any combination thereof.

11. An activation medium for preparing an activated frozen cell transfection system (AFCT-system) that is configured for transfection of a nucleic acid into a eukaryotic cell immediately upon thaw and without a pre-culture step comprising:
a cell transfection medium with a cryopreservant overload ratio; and
a eukaryotic cell for combination with said cell transfection medium to produce cell-supplemented cell transfection medium,
wherein the cell transfection medium combined with the eukaryotic cell are ready for freezing.

12. The activation medium of claim 11, wherein the nucleic acid is purified, unencumbered exogenous nucleic acid, and wherein the medium is pre-conditioned with a positively charged polymer that is not associated with nucleic acid and is capable of mediating transfection of the purified, unencumbered exogenous nucleic acid into the eukaryotic cell.

13. The activation medium of claim 11, wherein said cell transfection medium comprises:
5-200 mg/L CaCl₂) (anhydrous);
15-70 mg/L MgCl₂ (anhydrous);
0-0.08 mg/L Fe(NO₃)₃ 9H₂O;
20-110 mg/L MgSO₄ (anhydrous);
30-100 mg/L Na₂HPO₄;
30-300 mg/L NaH₂PO₄ H₂O;
0.002-0.07 mg/L SeNa₂O₃;
280-500 mg/L KCl;
40-1050 mg/L L-Asparagine H₂O;
20-1000 mg/L L-Aspartic acid;
50-1000 mg/L L-Isoleucine;
50-1200 mg/L L-Leucine;
50-500 mg/L L-Methionine;
100-1000 mg/L L-Valine;
25-1000 mg/L L-Phenylalanine;
25-430 mg/L L-Tyrosine, 2Na, 2H₂O;
100-1200 mg/L L-Lysine HCl;
50-1050 mg/L L-Threonine;
100-500 mg/L L-Histidine;
50-500 mg/L L-Serine;
2-500 mg/L L-Tryptophan;
200-5000 mg/L L-Arginine HCl;
25-250 mg/L L-Cysteine;
15-150 mg/L L-Cysteine 2HCl;
0.003-1 mg/L D-Biotin;
0.05-5 mg/L Vitamin B12;
0.05-5 mg/L Riboflavin;
0.5-20 mg/L Thiamine HCl;
0.1-7 mg/L D-calcium pantothenate;
0.5-30 mg/L Pyridoxine HCl;
1-20 mg/L Folic acid;
1-150 mg/L Choline chloride;
10-1000 mg/L Myo-inositol;
2-100 mg/L Ethanolamine HCl;
0.025-6 mg/L Putrescine 2HCl;
0.03-1 mg/L DL-α-lipoic acid;
0.01-2 mg/L Linoleic acid;
500-8000 mg/L D-Glucose;
0.001-0.02 mg/L CuSO₄ 5H₂O;
0-2 mg/L FeSO₄ 7H₂O;
0.4-2 mg/L ZnSO₄ 7H₂O;
0.00007-4.5 mg/L MnSO₄ H₂O;
5000-7500 mg/L NaCl;
0-1000 mg/L L-Proline;
0-1000 mg/L L-Glutamic acid;
0-500 mg/L Glycine;
0-1000 mg/L Sodium pyruvate;
0-20 mg/L Hypoxanthine in NaOH 1M;
0-3 mg/L Thymidine in NaOH 1M;
0-150 mg/L L-Alanine;
0-100 mg/L beta-Alanine;
0-100 mg/L L-Ornithine;
0-1000 mg/L L-Taurine;
0.9-1.1 mg/L L-α-phosphatidylcholine;
0.009-0.011 mg/L Hydrocortisone;
5300-6600 mg/L HEPES;
0-1100 mg/L Lutrol® or Pluronic® F-68;
0-7 mg/L Iron gluconate, 2H₂O;
0-200 mg/L Ferric ammonium citrate;
0.001-0.10 mg/L CoCl₂ 6H₂O;
0.001-0.005 mg/L (NH₄)₆Mo₇O₂₆ 4H₂O;
0.000025-0.0005 mg/L NiSO₄ 6H₂O;
0.02-0.4 mg/L Na₂SiO₃ 9H₂O;
0.000025-0.0005 mg/L SnCl₂ 2H₂O;
0.0001-0.0025 mg/L NH₄VO₃;
0.5-30 mg/L Nicotinamide (B3);
0.1-20 mg/L p-aminobenzoic acid;
500-650 mg/L L-Glutamine;
2000-2200 mg/L NaHCO₃;
0-110 mg/L Ferric citrate;
0-20000 mg/L Plant hydrolysates;
0-20000 mg/L Animal hydrolysates;
0-10% mg/L Serum, or any combination thereof.

14. The activation medium of claim 12, wherein said positively-charge polymer is polyethylenimine.

15. The activation medium of claim 11, wherein said eukaryotic cell is selected from the group consisting of a mammalian cell, a fish cell, an insect cell, an avian cell, metazoan cell, and any combination thereof.

16. The activation medium of claim 11, wherein said nucleic acid is selected from the group consisting of DNA, RNA, PNA, LNA, and any combination thereof.

17. A method of transfecting eukaryotic cells comprising:
obtaining an activated frozen cell transfection system that is configured for transfection of a nucleic acid into a eukaryotic cell immediately upon thaw and without a pre-culture step comprising:
a cell transfection medium with a cryopreservant overload ratio; and
a eukaryotic cell for combination with said cell transfection medium to produce cell-supplemented cell transfection medium,
wherein the cell transfection medium combined with the eukaryotic cell are frozen;
thawing the activated frozen cell transfection system;
combining the thawed activated frozen cell transfection system with exogenous nucleic acid without a pre-culture step,
such that said exogenous nucleic acid is transfected into said eukaryotic cells immediately upon thaw and without a pre-culture step.

18. The method of claim 17, wherein the nucleic acid is a purified, unencumbered exogenous nucleic acid.

19. A kit for transfection of eukaryotic cells comprising:
an activated frozen cell transfection system (AFCT-system) of claim 1; and
a nucleic acid that is a DNA vector compatible with said eukaryotic cells.

20. The kit of claim 19, wherein said eukaryotic cells are mammalian cells.
